(19) United States
(12) Patent Application Publication
(10) Pub. No.: US 2023/0076470 A1
(43) Pub. Date: Mar. 9, 2023

(54) Heart valve prosthesis

(71) Applicant: BIOTRONIK AG, Buelach (CH)

(72) Inventor: Boris Lux, Hamburg (DE)

(21) Appl. No.: 17/799,832

(22) PCT Filed: Feb. 12, 2021
(86) PCT No.: PCT/EP2021/053434

§ 371 Date: Aug. 15, 2022

(30) Foreign Application Priority Data
Mar. 2, 2020 (EP) 20160336.2

(51) Int. Cl. A61F 2/24

(52) U.S. Cl. CPC A61F 2/2418; A61F 2/2415; A61F 2230/0067; A61F 2250/0039; A61F 2250/006

(57) ABSTRACT

A heart valve prosthesis includes a valve member having a flexible leaflet arrangement and a valvular support structure to support the flexible leaflet arrangement. The flexible leaflet arrangement includes a plurality of flexible leaflets. Each flexible leaflet forms a free edge. The free edges are configured to abut one another in a closed position to block blood flow in a retrograde flow direction. When viewed in a cross-sectional plane transverse to the antegrade flow direction, a first of the flexible leaflets extends across a first opening angle and a second of the flexible leaflets extends across a second opening angle different from the first opening angle. The free edge the first of the flexible leaflets has a first convex curvature and the free edge of the second of the flexible leaflets has a second convex curvature different than the first convex curvature.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2021/053434, which was filed Feb. 12, 2021, which application claimed priority from European Application Serial Number 20160336.2, which was filed Mar. 2, 2020.

### FIELD OF THE INVENTION

The field of the invention is heart valve prostheses.

### BACKGROUND

A known heart valve prosthesis includes a valve member having a flexible leaflet arrangement and a valvular support structure for supporting the flexible leaflet arrangement. The flexible leaflet arrangement includes a plurality of flexible leaflets which are movable between an opened position for allowing a blood flow in an antegrade flow direction and a closed position for blocking a blood flow in a retrograde flow direction. Each flexible leaflet forms a free edge, the free edges of the flexible leaflets being configured to about one another in the closed position for blocking a blood flow in the retrograde flow direction.

In typical heart valve prosthesis arrangements, as known for example from U.S. Pat. No. 9,750,603 B2 a flexible leaflet arrangement, for example formed from a pericardial material, is fixed to a support structure in the shape of a stent. The stent forms a lattice structure having a tubular shape and includes a plurality of closed cells arranged to define a band exhibiting a variable radial stiffness, the flexible leaflet arrangement being placed within a lumen of the stent. The stent herein is formed to self-expand, such that the stent may be delivered to a location of interest in a compressed state, and may expand upon deployment at the location of interest, in particular at the location of the aortic valve within a patient's heart.

DE 10 2017 202 159 A1 describes a heart valve prosthesis that allows for an implantation using a delivery catheter and includes a flexible, foldable support structure to which valve leaflets are fixed. The support structure includes a fixing ring which force-decouples from the support structure such that a deformation of the fixing ring is possible without a deformation of the support structure upon implantation within a blood vessel of a patient.

WO 2005/072654 A1 describes an anatomically approximate prosthetic heart valve including dissimilar flexible leaflets, dissimilar commissures and/or a non-circular flow orifice. The heart valve may be implanted in the mural position and has one larger leaflet oriented along the anterior aspect to mimic the natural anterior leaflet.

### SUMMARY OF THE INVENTION

A preferred embodiment is a heart valve prosthesis that includes a valvular support structure and a plurality of flexible leaflets supported by the valvular support structure. The flexible leaflets are movable between an opened position to permit a blood flow in an antegrade flow direction (F) and a closed position to block a blood flow in a retrograde flow direction (R). Each flexible leaflet forms a free edge, and the free edges are configured to abut one another in the closed position. When viewed in a cross-sectional plane transverse to the antegrade flow direction (F), a first of the flexible leaflets extends across a first opening angle (β_{A}, β_{B}) and a second of the flexible leaflets extends across a second opening angle (β_{C}) different from the first opening angle (β_{A}, β_{B}). The free edge of the first of the flexible leaflets includes a first convex curvature and the free edge of the second of the flexible leaflets includes a second convex curvature different than the first convex curvature.

The prosthesis can include an anchoring member that has a flexible skirt and an anchoring support structure that supports the flexible skirt. The valvular support structure and the anchoring support structure are structurally separate from one another, but are flexibly connected to each other via the flexible leaflets and the flexible skirt.

### BRIEF DESCRIPTION OF THE DRAWINGS

The object of the invention shall subsequently be explained in more detail with reference to the embodiments shown in the figures. Herein:

FIG. 1 shows a drawing of an embodiment of a heart valve prosthesis including a valve member and an anchoring member;

FIG. 2 shows a drawing of the valve member and the anchoring member in a separate state;

FIG. 3 shows a separate view of the valve member;

FIG. 4 shows a view of a valvular support structure of the valve member;

FIG. 5 shows a view of the valve member, in a closed position of leaflets of a flexible leaflet arrangement;

FIG. 6 shows a separate view of the anchoring member;

FIG. 7 shows a view of an anchoring support structure of the anchoring member;

FIG. 8 shows a view of an embodiment of a piece of material forming the flexible leaflet arrangement of the valve member;

FIG. 9 shows a view of an embodiment of a piece of material forming a single leaflet of the flexible leaflet arrangement;

FIG. 10 shows a view of a fastening section of the flexible leaflet arrangement;

FIG. 11 shows an embodiment of a piece of material forming the flexible skirt arrangement;

FIG. 12 shows a view of an embodiment of the anchoring member in the region of an inflow edge illustrating the fixation of the skirt arrangement to the anchoring support structure;

FIG. 13 shows another view of the embodiment of the anchoring member according to FIG. 12;

FIG. 14 shows a schematic view of the anchoring member of the embodiment of FIG. 12;

FIG. 15 shows a schematic view of the anchoring member, illustrating a force distribution in an implanted state of the anchoring member;

FIG. 16A shows a view of a connection of wire ends of a wire forming an anchoring support structure or a valvular support structure;

FIG. 16B shows a view of another embodiment of a connection of wire ends of a wire forming an anchoring support structure or a valvular support structure;

FIG. 17 shows a view of an embodiment of a valve member, illustrating seams for closing the flexible leaflet arrangement and the flexible skirt arrangement as well as for connecting the flexible leaflet arrangement and the flexible skirt arrangement to each other;

FIG. 18 shows a view of an embodiment of a piece of material for forming the flexible leaflet arrangement;

FIG. 19 shows a view of joining edges of the flexible leaflet arrangement and the flexible skirt arrangement for interconnecting the flexible leaflet arrangement and the flexible skirt arrangement;

FIG. 20 shows a view of the joining edges in an interconnected state, from inside the valve;

FIG. 21 shows a view of a seam for interconnecting the flexible skirt arrangement and the flexible leaflet arrangement;

FIG. 22 shows another view of the seam, from outside the valve;

FIG. 23 shows yet another view of the seam;

FIG. 24 illustrates a step for manufacturing a valve member;

FIG. 25 shows a view of the flexible leaflet arrangement of the valve member during manufacturing;

FIG. 26 illustrates the placement of a valvular support structure within the flexible leaflet arrangement;

FIG. 27 illustrates the fixing of the flexible leaflet arrangement to the valvular support structure;

FIG. 28 further illustrates the fixing of the flexible leaflet arrangement to the valvular support structure;

FIG. 29 provides a side view of the arrangement of FIG. 28;

FIG. 30 illustrates the flexible leaflet arrangement fixed to the valvular support structure;

FIG. 31 illustrates a sewing of a fastening section to the valvular support structure;

FIG. 32 shows a schematic view of the flexible leaflet arrangement fixed to the valvular support structure;

FIG. 33 shows a step in manufacturing an anchoring member;

FIG. 34 illustrates another step in manufacturing the anchoring member;

FIG. 35 illustrates the fixing of a flexible skirt arrangement of the anchoring member to an anchoring support structure;

FIG. 36 illustrates a seam for fixing the anchoring support structure to the flexible skirt arrangement;

FIG. 37 shows a schematic view of the seam for connecting the flexible skirt arrangement and the anchoring support structure;

FIG. 38 shows another step in manufacturing the anchoring members;

FIG. 39 shows the anchoring member prior to connecting it to the valve member;

FIG. 40 illustrates the interconnection in between the flexible skirt arrangement and the anchoring support structure;

FIG. 41 shows the first heart valve prosthesis with a valve member connected to the anchoring member;

FIG. 42 a view of an embodiment of a valve member of a heart valve prosthesis including leaflets;

FIGS. 43A-C views of leaflets differing in shape to make up a flexible leaflet arrangement of the valve member of FIG. 42;

FIG. 44 a schematic view of an asymmetric valve member of a heart valve prosthesis in accordance with the instant invention;

FIG. 45 a schematic view of opening angles of leaflets making up the valve member;

FIG. 46 a schematic view of leaflets for forming a conical leaflet arrangement; and

FIG. 47 a schematic view of a conical leaflet arrangement for forming a valve member.

### DETAILED DESCRIPTION OF THE PREFERRED EMBODIMENTS

A preferred embodiment is a heart valve prosthesis that includes a valvular support structure and a plurality of flexible leaflets supported by the valvular support structure. The flexible leaflets are movable between an opened position to permit a blood flow in an antegrade flow direction (F) and a closed position to block a blood flow in a retrograde flow direction (R). Each flexible leaflet forms a free edge, and the free edges are configured to abut one another in the closed position. Accordingly, when viewed in a cross-sectional plane transverse to the antegrade flow direction, a first of the flexible leaflets extends across a first opening angle and a second of the flexible leaflets extends across a second opening angle different from the first opening angle, wherein the free edge of said first of the flexible leaflets includes a first convex curvature and the free edge of said second of the flexible leaflets includes a second convex curvature different than the first convex curvature.

The heart valve prosthesis shall be implanted in a patient, for example in the aorta of the patient, in order to replace the natural aortic valve of the patient. In contrast to a conventional heart valve prosthesis including a generally circular cross section, the proposed heart valve prosthesis uses leaflets which have a different geometric shape and hence may cause the heart valve prosthesis, in particular in a plane of the leaflets and an orifice formed by the leaflets in the opened position of the heart valve prosthesis, to have an asymmetric, noncircular shape. This allows for a variable adaption of the heart valve prosthesis to the actual shape at the implantation site at which the heart valve prosthesis shall be placed, having the potential to improve operation of the heart valve prosthesis upon implantation.

In particular, a first of the flexible leaflets extends across a first opening angle, and a second of the flexible leaflets extends across a different, second opening angle. Hence, when viewed in a cross-sectional plane transverse to the antegrade flow direction, the leaflets span different opening angles, which are generally measured between fastening sections at which the leaflets are connected to the valvular support structure in between which the free edges of the leaflets extend. If the flexible leaflet arrangement is formed by multiple pieces of material providing the leaflets, the opening angle of a leaflet may correspond to the angle in the cross-sectional plane between seams at which the single pieces forming the leaflets are sewed together.

The flexible leaflets hence span different angles, causing a different shape of the leaflets.

In one embodiment, the second opening angle is smaller than the first opening angle.

In one embodiment, the heart valve prosthesis includes two first flexible leaflets each extending across said first opening angle, and a single second flexible leaflet extending across said second opening angle. In another embodiment, more than two first flexible leaflets are present, and one second flexible leaflet. In yet another embodiment, two or more first flexible leaflets and two or more second flexible leaflets are present.

In addition to the different shaping of the flexible leaflets by having the flexible leaflets extend across different opening angles, the free edges of the flexible leaflets include different curvatures. In particular, the first of the flexible leaflets includes a first convex curvature at its free edge, and the second of the flexible leaflets includes a second convex curvature at its free edge different than the first convex curvature. Each free edge hence is curved in a convex manner. However, the curvature of the different leaflets differs, for example in that the second convex curvature is smaller than the first convex curvature.

The convex curvature may in particular be measured at a center point which is located on the free edge in the middle in between fastening sections bounding the free edge of each leaflet. At the fastening sections the flexible leaflet arrangement is fixed to the valvular support structure. Each leaflet herein at its free edge includes a convex curvature in particular in a middle region in between two bounding, outer fastening sections.

The curvature of the free edge may in particular be defined by a radius of curvature, in particular a radius of curvature, at a center point of the corresponding free edge. A large curvature herein is defined by a small radius of curvature, whereas in contrast a small curvature is defined by a large radius of curvature. Hence, the free edge of the first of the flexible leaflets may for example include a small radius of curvature, whereas the free edge of the second of the flexible leaflets includes a larger, second radius of curvature, measured in particular at the center point of the corresponding free edge.

By shaping the free edges of the different leaflets differently and by in particular providing a different curvature at the free edges, the valve member, in particular in the plane of the flexible leaflets, may have a shape diverting from a circular shape, for example an oval (elliptical) or otherwise non-symmetric shape, when viewed in a cross-sectional plane perpendicular to the antegrade flow direction. This allows to flexibly adapt the heart valve prosthesis to the shape of the aorta, such that placement and function of the heart valve prosthesis at an implantation site may be improved.

In the closed state, herein, the free edges of the leaflets abut one another, wherein the different leaflets are connected and arranged with respect to one another such that the free edges, in the closed position, effectively close an orifice formed within the heart valve prosthesis in order to block a retrograde blood flow.

In one embodiment, the flexible leaflets are formed from separate pieces of material, wherein the geometric shape of the piece of material forming said first of the flexible leaflets differs from the geometric shape of the piece of material forming said second of the flexible leaflets. For forming the flexible leaflet arrangement of the valve member, the single pieces of material are sewed together. In an assembled state, herein, due to the different geometric shape of the pieces of material the flexible leaflet arrangement assumes an asymmetric shape, the first and second leaflets in particular differing in their opening angle.

The flexible leaflet arrangement may, in another embodiment, be formed from a single, integral piece of material forming all leaflets.

In one embodiment, the flexible leaflet arrangement may be conically tapered in the retrograde flow direction. Hence, the valve member, due to the shaping of the leaflet arrangement, assumes a conical shape, the valve member being tapered in the retrograde flow direction and hence towards an inflow edge of the heart valve prosthesis.

The conical shape of the valve member may provide for a beneficial force introduction and support in particular in a closed position of the leaflet arrangement for blocking a flow in the retrograde flow direction. In case of a retrograde flow, the leaflets of the leaflet arrangement are moved from an opened position into a closed position, in which the leaflets are flexibly bent inwards and abut each other in order to close a flow path. Due to the conical shape of the valve member, herein, a deformation of the valve member, in particular the valvular support structure (which may be pulled inwards due to forces acting onto the leaflets of the leaflet arrangement) is reduced, the conical shape providing for a structurally beneficial force introduction and a reduced load acting onto the valvular support structure.

In addition, the valve member may be to some extent deformed by the deployment at the implantation site, for example within the aorta, such that the valvular support structure is partially compressed due to the placement at the implantation site. Such deformation may cause a drapery in the leaflet arrangement. In case of a blocking of flow in the retrograde flow direction in the closed position of the leaflets of the leaflet arrangement, a loading on the valve member first causes a reduction in the drapery in the leaflet arrangement, before a substantial load acts onto the valvular support structure.

In one aspect, the valvular support structure is formed by a ring. The ring may be circumferentially closed or may be opened at a circumferential location.

The ring may be formed from a wire. The wire may be circumferentially closed, the ring hence being formed by a closed loop. Alternatively, the wire forming the ring may have two ends, wherein the ring may be opened at one circumferential position, or the ends of the wire may be fixedly connected to each other such that a closed loop is formed.

In one embodiment, the ring is formed by a continuous element having no branch-offs. The valvular support structure hence does not form any cells and does not form any nodes from which multiple (more than two) strut sections extend.

In one embodiment, the valvular support structure may have a meandering or crown-like shape, the valvular support structure being bent such that upper vertices alternate with lower vertices, the upper vertices being arranged in the vicinity of an outflow end of the valve member, wherein the lower vertices point towards the inflow.

The valvular support structure, in one aspect, may have a conical shape, the lower vertices being displaced radially inwards with respect to the upper vertices.

In one aspect, the valvular support structure forms a plurality of curved sections for supporting the flexible leaflet arrangement, the curved sections being associated with the is flexible leaflets for defining a bending line about which the flexible leaflets are flexibly bendable for a movement between the opened position and the closed position. The number of curved sections of the valvular support structure, in one embodiment, matches the number of leaflets of the leaflet arrangement. In particular, each leaflet of the leaflet arrangement may be associated with a curved section of the valvular support structure, such that each curved section defines a bending line for the associated leaflet of the flexible leaflet arrangement.

Herein, the curved sections may for example have a parabolic shape, each curved section extending in between a pair of upper vertices of the valvular support structure and including a lower vertex arranged in between the pair of upper vertices. Each curved section, hence, with its lower vertex, points towards the inflow.

In one aspect, the valvular support structure forms at least one tip section, wherein the flexible leaflet arrangement is fixed to the at least one tip section. The number of tip sections formed by the valvular support structure may for example match the number of leaflets of the flexible leaflet arrangement. A tip section herein may for example be formed in between two adjacent leaflets of the leaflet arrangement. For example, the tip section may be formed at the location of an upper vertex of the valvular support structure, the valvular support structure for example having a meandering or crown-like shape.

The valvular support structure may include one tip section, but may, in another embodiment, also include more than one tip section. For example, the valvular support structure may include 2, 3, 4 or 5 tip sections, wherein it also is conceivable that the valvular support structure includes more than 5 tip sections.

The number of tip sections may, in one embodiment, correspond to the number of leaflets of the flexible leaflet structure.

The fixing of the flexible leaflet arrangement to the at least one tip section of the valvular support structure may take place in different ways.

For example, the flexible leaflet arrangement may be tightly hang-up on the at least one tip section. In this case, for example, a fastening section in the shape of a bag-like structure is formed on the flexible leaflet arrangement, the tip section engaging with the bag-like structure such that the flexible leaflet arrangement is hung up on the tip sections of the valvular support structure.

In another embodiment, the flexible leaflet arrangement forms at least one fastening section adjacent, when viewed along a circumferential direction about the antegrade flow direction, to one of the flexible leaflets of the leaflet arrangement, wherein the flexible leaflet arrangement is fixed to the tip section via the at least one fastening section.

The at least one fastening section may, for example, include at least one flap section which, for fixing the flexible leaflet arrangement to the tip section, is folded about a beam section of the valvular support structure at the tip section, wherein for example two flap sections of the at least one fastening section may be sewed together in the vicinity of the tip section for fixing the flexible leaflet arrangement to the tip section.

In one embodiment, the heart valve prosthesis includes an anchoring member for anchoring the heart valve prosthesis within a patient, the anchoring member including a flexible skirt arrangement and an anchoring support structure for supporting the flexible skirt arrangement. Herein, the valvular support structure and the anchoring support structure are structurally separate from one another but are flexibly connected to each other via the flexible leaflet arrangement and the flexible skirt arrangement.

According to this embodiment, a valve member having a valvular support structure and an anchoring member having an anchoring support structure are provided. The valvular support structure and the anchoring support structure herein are structurally separate from one another, such that the valvular support structure and the anchoring support structure are formed by different structural units and as such are not structurally interconnected with each other.

is Within the context of the instant text the formation of the valvular support structure and the anchoring support structure as structurally separate units shall be understood to mean that no rigid structural features, such as struts or the like, are formed in between the valvular support structure and the anchoring support structure. The valvular support structure and the anchoring support structure hence are not interconnected with each other by structural elements such as struts, beams or the like extending in between the valvular support structure and the anchoring support structure.

However, a flexible interconnection between the valvular support structure and the anchoring support structure is formed via the flexible leaflet arrangement and the flexible skirt arrangement. In particular, the flexible leaflet arrangement of the valve member and the flexible skirt arrangement of the anchoring member are connected to each other, the flexible leaflet arrangement and the flexible skirt arrangement being formed from a flexible, soft, deformable, pliable material such that by the flexible leaflet arrangement and the flexible skirt arrangement no structural interconnection, but a flexible, pliable interconnection between the valvular support structure and the anchoring support structure is formed.

The flexible leaflet arrangement of the valve member and the flexible skirt arrangement of the anchoring member together may be formed integrally in one piece from a pliable material. In another embodiment, the flexible leaflet arrangement of the valve member and the flexible skirt arrangement of the anchoring member may be formed as separate members and may for example be sewed together for providing an interconnection in between the valve member and the anchoring member.

Because the valvular support structure of the valve member and the anchoring support structure of the anchoring member are formed by separate frame-like structures, the heart valve prosthesis upon implantation within a patient may flexibly adapt to specific conditions at a location of interest, for example in the vicinity of the aortic valve and within the aorta. In particular, the positions of the valvular support structure and the anchoring support structure may flexibly be adapted relative to each other, without the anchoring support structure constraining the positioning of the valvular support structure and vice versa. In this way, the anchoring support structure may in particular provide for a fixation to the annulus of an aortic valve, wherein the valvular support structure may be placed within the aorta and may adapted to the specific shape of the aorta.

The heart valve prosthesis, hence, upon implantation, may flexibly adapt to conditions at an implantation site within a patient.

In addition, because the valve member and the anchoring member are formed with separate support structures, delivery may be eased in that, e.g., a delivery catheter may have a reduced bending stiffness and hence may be passed through the aortic bend in an easy manner.

During delivery, the placement of the anchoring member and the valve member may take place in a multi-step process, in which, e.g., first the anchoring member is deployed and placed for example at the annulus of the aortic valve and, e.g., in a second step the valve member is deployed by placing the valve member within the aorta of the patient.

In one aspect, the flexible leaflet arrangement and/or the flexible skirt arrangement are made from a biocompatible material, in particular from a biocompatible pericardial material of animal origin (e.g. a dry biocompatible pericardial material), preferably a bovine, porcine, horse or kangaroo pericardial material, from a material based on bacterial cellulose, or from a polymeric material. Most preferred may be, in one embodiment, a porcine pericardial material; e.g. a dry porcine pericardial material.

The flexible leaflet arrangement and the flexible skirt arrangement, herein, may be made from the same material, for example from a pericardial material or a polymeric material. Alternatively, the flexible leaflet arrangement and the flexible skirt arrangement may be made from different materials, one for example being formed from a pericardial material and the other from a polymeric material. The latter allows for an adaption of the leaflet arrangement and the skirt arrangement to specific needs and conditions, in particular to a specific arrangement, for example the leaflet arrangement, to requirements for a flexibility, uniformity and elasticity, the skirt arrangement potentially having a reduced requirement in this respect.

In one aspect, the flexible skirt arrangement forms an inflow edge, wherein at least a portion of the flexible skirt arrangement is conically tapered in the antegrade flow direction beyond the inflow edge. The anchoring member, hence, due to the shaping of the skirt arrangement, assumes a conical shape, the anchoring member tapering in the antegrade flow direction towards the valve member.

In one aspect, both the anchoring member and the valve member have a conical shape, the skirt arrangement of the anchoring member and the leaflet arrangement of the valve member being connected to each other at a waist of the heart valve prosthesis, the waist corresponding to a location, when viewed along the antegrade flow direction, at which the heart valve prosthesis includes a minimum diameter (measured transverse to the antegrade flow direction).

In one aspect, the anchoring support structure is formed by a ring. The ring may be circumferentially closed or may be opened at a circumferential location.

The ring may be formed from a wire. The wire may be circumferentially closed, the ring hence being formed by a closed loop. Alternatively, the wire forming the ring may have two ends, wherein the ring may be opened at one circumferential position, or the ends of the wire may be fixedly connected to each other such that a closed loop is formed.

In one embodiment, the ring forming the anchoring support structure is formed by a continuous element having no branch-offs. The anchoring support structure hence does not form any cells and does not form any nodes from which multiple (more than two) strut sections extend.

In one aspect, the anchoring support structure may have a meandering shape, upper vertices and lower vertices of the anchoring support structure hence alternating with each other, the upper vertices being displaced with respect to the lower vertices along the antegrade flow direction.

In one aspect, the anchoring support structure includes a plurality of radially outer portions and a plurality of radially inner portions, wherein, when viewed along a circumferential direction about the antegrade flow direction, the radially outer portions alternate with the radially inner portions. The radially outer portions are displaced with respect to the radially inner portions radially towards the outside.

The radially outer portions, in particular, may serve to provide for an abutment to native structure of a patient, for example in the region of the annulus of the aortic valve of the patient. Hence, by the radially outer portions a fixation of the anchoring member to native structure or, possibly, to a prior prosthetic valve which shall be replaced by the heart valve prosthesis is provided.

The radially inner portions, in particular, may serve for fixing the skirt arrangement of the anchoring member to the anchoring support structure, the skirt arrangement for example being sewed to the radially inner portions of the anchoring support structure.

The flexible skirt arrangement, in one embodiment, may for example be fixed to the anchoring support structure by a seam circumferentially extending about the anchoring support structure.

In one embodiment, the plurality of radially outer portions is displaced, along the antegrade flow direction, with respect to said plurality of radially inner portions. The radially outer portions may for example include upper vertices of the anchoring support structure, the radially inner portions in turn including lower vertices of the anchoring support structure.

to In one aspect, the flexible skirt arrangement includes a body and at least one folding section formed on the body. The at least one folding section is folded, at a fold line, with respect to the body to reach around the anchoring support structure. The at least one folding section may for example have a flap-like shape, the at least one folding section being folded with respect to the body of the skirt arrangement such that the at least one folding section reaches around the anchoring support structure, for example in the vicinity of a lower vertex of the anchoring support structure, for fixing the flexible skirt arrangement to the anchoring support structure.

In one embodiment, the flexible skirt arrangement includes multiple folding sections, each of the folding sections being associated with a lower vertex of the anchoring support structure and being folded around an associated lower vertex of the anchoring support structure for fixing the skirt arrangement circumferentially to the anchoring support structure.

In one embodiment, the at least one folding section may be folded with respect to the body such that the at least one folding section is placed radially outside of the body.

In one aspect, the flexible leaflet arrangement includes a first joining edge and the flexible skirt arrangement includes a second joining edge, wherein the first joining edge and the second joining edge are connected to each other by a seam. The flexible leaflet arrangement and the flexible skirt arrangement are formed, in one embodiment, from separate units of flexible, pliable material, the flexible leaflet arrangement and the flexible skirt arrangement being interconnected with each other by sewing the leaflet arrangement and the skirt arrangement together.

In one embodiment, the first joining edge and/or the second joining edge include a meandering shape, joining flaps alternating with recesses along a circumferential direction about the antegrade flow direction. In case both the first joining edge and the second joining edge include a meandering shape, joining flaps of the first joining edge and joining flaps of the second joining edge may be arranged with respect to each other such that the joining flaps of the first joining edge and the joining flaps of the second joining edge interleave with each other. The joining flaps, in one embodiment, may point towards the outside, such that the joining flaps are arranged radially outside of a body of the skirt arrangement and a body of the flexible leaflet arrangement.

In one aspect, the seam for interconnecting the flexible skirt arrangement and the flexible leaflet arrangement to each other is formed by at least one thread forming first thread sections extending between neighbouring first joining flaps, and forming second thread sections extending between neighbouring second joining flaps.

In one embodiment, said first thread sections and said second thread sections are arranged substantially parallel to one another.

In one embodiment, said first thread sections and said second thread sections are displaced with respect to each other along the antegrade flow direction.

In one embodiment, said first thread sections and said second thread sections are arranged at a side of the heart valve prosthesis facing radially outwards.

In one embodiment, the at least one thread forms third thread sections and fourth thread sections arranged at an angle with respect to one another. Said third thread sections and said fourth thread sections, in one embodiment, are arranged at a side of the heart valve prosthesis facing radially outwards.

In particular, the entire thread included of first, second, third and fourth thread sections may be placed outside of an inner lumen of the heart valve prosthesis, such that the thread does not interfere with a blood flow though the inner lumen.

In one embodiment, at least some of the first thread sections are adjoined by a third thread section and a fourth thread section.

In one embodiment, at least some of the second thread sections are adjoined by a third thread section and a fourth thread section.

The thread sections may together form a seam having a substantially trapezoidal shape, the thread sections forming trapezoids being for example opened and having one or two edges. Because in one embodiment the thread sections are arranged at an outside of the heart valve prosthesis, at which also for example the joining flaps of the flexible leaflet arrangement and the flexible skirt arrangement come to rest, the seam may be formed such that the joining flaps come to lie flatly about a body of the flexible leaflet arrangement respectively a body of the flexible skirt arrangement.

In one aspect, the connection of the valvular support structure and the anchoring support structure via the flexible leaflet arrangement and the flexible skirt arrangement is releasable in an implanted state of the heart valve prosthesis. By providing a releasable connection in between the valvular support structure and the anchoring support structure, it may be achieved that the valve member and the anchoring member may be disconnected from each other for example for replacing the valve member in an implanted state of the heart valve prosthesis. In particular, in case of a failure or in case of another replacement need the valve member may be disconnected from the anchoring member and hence may be replaced by a replacement valve member while the anchoring member remains in place within the patient.

The releasable connection of the valve member and the anchoring member also makes it possible that for implantation, for example, first the anchoring member and, in another implantation step, the valve member is implanted, wherein the anchoring member and the valve member are connected to each other only within the patient, and hence while, for example, the anchoring member already is implanted.

The connection in between the valve member and the anchoring member, that is in between the valvular support structure of the valve member and the anchoring support structure of the anchoring member, is achieved via the flexible leaflet arrangement and the flexible skirt arrangement. For achieving a releasable connection, herein, for example joining flaps at the meander-shaped joining edges of the flexible leaflet arrangement and the flexible skirt arrangement may be brought into engagement with each other for connecting the flexible leaflet arrangement and the flexible skirt arrangement to each other, wherein the connection may be releasable by releasing the engagement in between the joining flaps; for example, by exerting a predefined movement in between the valve member and the anchoring member.

The joining flaps, for example, may provide for a connection in the manner of a bayonet lock.

The joining flaps may be formed in a rigid manner or may include rigid sections such that a reliable, positive locking connection may be achieved via the joining flaps. To provide for rigid sections, for example rigid elements may be sewed to the joining flaps to at least partially reinforce the joining flaps.

Alternatively or in addition, the joining flaps may be formed from a different material than the flexible leaflet arrangement and/or the flexible skirt arrangement, or may include a different material thickness, in particular an increased material thickness in comparison to other portions of the flexible leaflet arrangement and/or the flexible skirt arrangement. The material of the joining flaps in particular may provide for an increased rigidity in comparison to other portions of the flexible leaflet arrangement and/or the flexible skirt arrangement.

A releasable connection may also be provided by other means, for example by elements connected to the joining edges of the flexible leaflet arrangement and the flexible skirt arrangement, in particular locking elements providing for a positive locking connection.

Such locking elements may for example have a ring shape and may provide for a locking connection in the manner of a bayonet lock.

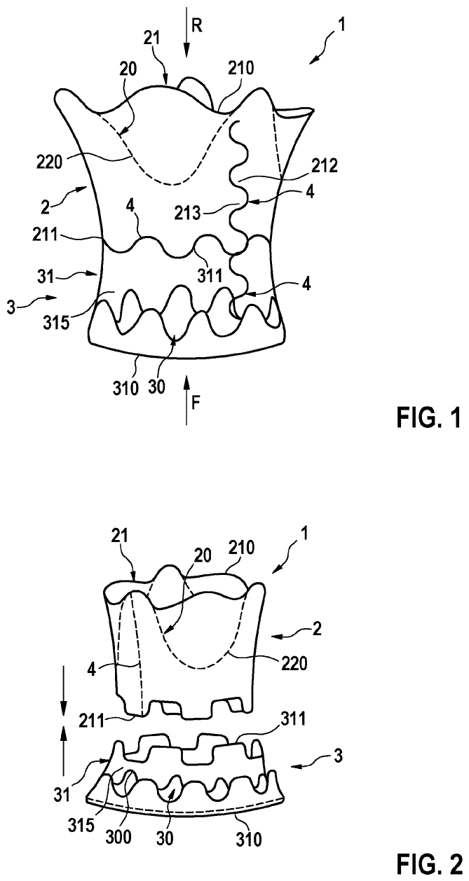

FIGS. 1 and 2 show an embodiment of a heart valve prosthesis 1 including a valve member 2 and an anchoring member 3.

The valve member 2 includes a flexible leaflet arrangement 21 supported by a valvular support structure 20. The anchoring member 3 includes a flexible skirt arrangement 31 supported by an anchoring support structure 30. The valve member 2 and the anchoring member 3 are interconnected with each other at joining edges 211, 311 of the flexible leaflet arrangement 21 and the flexible skirt arrangement 31 by a seam 4, such that a valve arrangement is formed allowing for a prosthetic replacement of a native heart of a patient, for example the aortic valve.

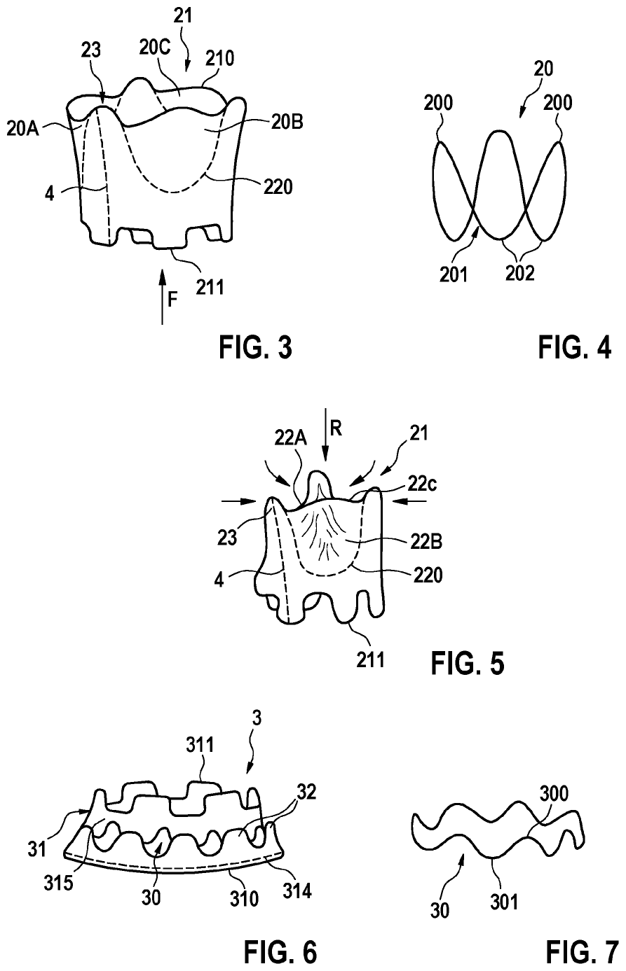

Referring now to FIGS. 3 to 5, the flexible leaflet arrangement 21 of the valve member 2 forms a plurality of leaflets 22A, 22B, 22C, which are flexibly movable with respect to the valvular support structure 20 for opening or closing the heart valve prosthesis 1. In particular, the leaflets 22A, 22B, 22C may assume an opened position in which the leaflets 22A, 22B, 22C are spaced apart from one another and form an opening therebetween, such that a flow may pass through the valve member 2 in an antegrade flow direction F, as illustrated in FIG. 3. In addition, the leaflets 22A, 22B, 22C may assume a closed position, as illustrated in FIG. 5, in which the leaflets 22A, 22B, 22C are moved to approach and abut one another, such that by the leaflets 22A, 22B, 22C a flow path in a reverse, retrograde flow direction R is closed and hence a flow passage is blocked by the heart valve prosthesis 1.

The valvular support structure 20, as illustrated in FIG. 4, has a meandering or crown-like shape and is received within the flexible leaflet arrangement 21 of the valve member 2. The valvular support structure 20 serves to structurally support the flexible leaflet arrangement 21, such that the leaflets are spatially spanned by the valvular support structure 20 and may flexibly move in between their opened position (FIG. 3) and their closed position (FIG. 5) in order to allow a flow in the antegrade flow direction F, but to reliably block a flow in the retrograde flow direction R.

As visible from FIG. 4, the valvular support structure 20 includes lower vertices 202 and upper vertices 200, which alternate along a circumferential direction about the antegrade flow direction F and the retrograde flow direction R. The upper vertices 200 are arranged in the vicinity of an outflow edge 210 formed by the flexible leaflet arrangement 21 and are displaced with respect to the lower vertices 202 along the antegrade flow direction F and the retrograde flow direction R. By the lower vertices 202, the valvular support structure 20 points towards the anchoring member 3.

The valvular support structure 20, in the shown embodiment, is formed by a ring which forms a circumferentially closed loop. The ring is circumferentially closed and does not include any branch-offs in that no nodes are formed by the ring from which more than two structural strut sections extend. Rather, the ring continuously extends in a meandering or crown-like shape to form the valvular support structure 20.

The valve member 2 includes a conical shape, the valve member 2 tapering in the retrograde flow direction R towards the anchoring member 3. The conical shape is defined by the shape of the flexible leaflet arrangement 21, which tapers towards the anchoring member 3. The conical shape in addition is defined by the shape of the valvular support structure 20, which likewise is tapered towards the anchoring member 3 in that the lower vertices 202 are displaced radially inwards with respect to the upper vertices 200, the valvular support structure 20 hence also tapering in the retrograde flow direction R.

Due to the conical shape of the valve member 2, a load acting onto the valve member 2 in the closed position of the leaflets 22A, 22B, 22C, caused by a retrograde flow acting onto the leaflet arrangement 21, may beneficially be taken up by the valvular support structure 20 and beneficially does not cause a substantial deformation of the valve member 2. In particular, due to the conical shape, a load force acting on the upper vertices 200 of the valvular support structure 20 by a pulling action of the flexible leaflet arrangement 21 is counteracted by the tapering shape of the valvular support structure 20.

The valvular support structure 20 forms curved sections 201 which may have a substantially parabolic shape and extend in between a pair of adjacent upper vertices 200. Each curved section 201 herein forms a respective lower vertex 202 arranged circumferentially in between the pair of associated upper vertices 200.

Each curved section 201 is associated with a leaflet 22A, 22B, 22C, the curved sections 201 defining bending lines 220 for the associated leaflets 22A, 22B, 22C, such that each leaflet 22A, 22B, 22C may bend in a defined manner about an associated bending line 220 when moving in between its opened position (FIG. 3) and the closed position (FIG. 5).

The valve member 2, upon implantation at an implantation site in a patient, may flexibly adapt to the specific conditions at the implantation site. In particular, the valvular support structure 20 may elastically deform to adapt to the shape of, for example, an aorta in which the valve member 2 is placed.

The valve member 2 is joined to the anchoring member 3 at the joining edges 211, 311 of the leaflet arrangement 21 and the skirt arrangement 31 by a seam 4. Herein, no structural interconnection in between the valvular support structure 20 and the anchoring support structure 30 exists, the valvular support structure 20 and the anchoring support structure 30 being formed by separate structural entities, with no structural features such as struts or the like extending in between the valvular support structure 20 and the anchoring support structure 30. A connection in between the valve member 2 and the anchoring member 3 is formed solely via the pliable material of the leaflet arrangement 21 and the skirt arrangement 31.

The flexible interconnection in between the valve member 2 and the anchoring member 3 via the pliable material of the leaflet arrangement 21 and the skirt arrangement 31 allows for a flexible, adaptable implantation of the valve member 2 and the anchoring member 3 at an implantation site, wherein the valve member 2 and the anchoring member 3 separately may adapt to specific conditions at the implantation site. In particular, the valvular support structure 20 and the anchoring support structure 30 may elastically deform independent of each other, such that the valvular support structure 20 may for example adapt to a specific shape of native structure within the aorta of a patient, whereas the anchoring member 3 may be placed for example within the annulus of an aortic valve of the patient, the anchoring support structure 30 being able to adapt to the specific shape of the annulus upon implantation.

In addition, due to the structurally separate formation of the valvular support structure 20 and the anchoring support structure 30, a delivery by a delivery catheter in a multi-step process becomes possible. In particular, the anchoring member 3 and the valve member 2 may be placed at an implantation site in different delivery steps, allowing for example to first deploy the anchoring member 3 within the annulus of a patient's aortic valve and to then, in another step, deploy the valve member 2 within the aorta of the patient.

Referring now to FIGS. 6 and 7, the anchoring member 3 is formed by the skirt arrangement 31 and the anchoring support structure 30 fixedly connected to the skirt arrangement 31. The anchoring support structure 30 includes a meandering shape having lower vertices 301 and upper vertices 300 which alternate along a circumferential direction about the antegrade flow direction F and the retrograde flow direction R. The anchoring support structure 30 is formed by a ring forming a circumferentially closed loop, the anchoring support structure 30 being fixed to the flexible skirt arrangement 31 in the region of the lower vertices 301, as this is visible from FIG. 7.

The flexible skirt arrangement 31 includes a body 315 and folding sections 32 having a flap-like shape. The folding sections 32 are folded about a fold line 314 with respect to the body 315 at an inflow edge 310 of the anchoring member 3, the folding sections 32 reaching around the anchoring support structure 32 at the lower vertices 301 of the anchoring support structure 30.

The folding sections 32 are folded with respect to the body 315 such that the folding sections 32 are placed radially outwards of the body 315, as visible from FIG. 6.

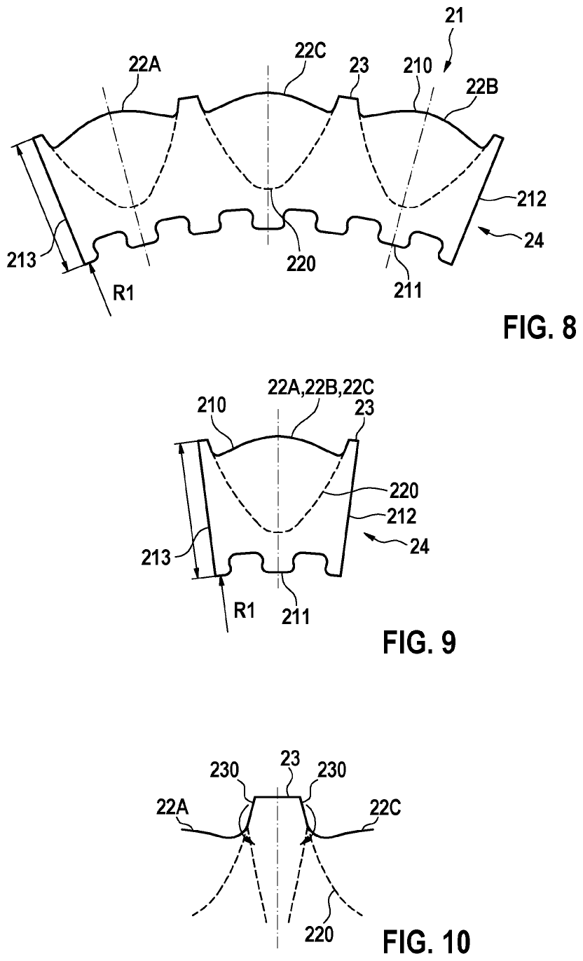

Referring now to FIG. 8, the flexible leaflet arrangement 21 of the valve member 2 may be formed by a single piece of a flexible, pliable material 24. In this embodiment, the leaflets 22A, 22B, 22C are formed from the same piece of material, wherein the flexible leaflet arrangement 21 is formed by sewing lateral edges 212, 213 of the piece of material 24 together to form a circumferentially closed structure, as visible from FIGS. 3 and 5.

In an alternative embodiment, the leaflets 22A, 22B, 22C may be formed from separate pieces of material 24, as illustrated in FIG. 9. Herein, the separate pieces 24 associated with the leaflets 22A, 22B, 22C are sewed together at edges 212, 213 to form a circumferentially closed structure in order to form a leaflet arrangement 21 similar to the one shown in FIGS. 3 and 5.

In each case, the material may be a biocompatible material, for example a biocompatible pericardial material of animal origin (e.g. a dry biocompatible pericardial material) such as a bovine, porcine, horse or kangaroo pericardial material, a material based on bacterial cellulose or a biocompatible polymeric material.

In one embodiment, independent on whether the leaflets 22A, 22B, 22C are formed from separate pieces or are formed from a single piece of material 24, the leaflets 22A, 22B, 22C may be pre-shaped such that they, e.g., do not have a flap shape, but may have a pre-shaped, curved shape. In this way, the leaflets 22A, 22B, 22C may, for example, form bags for trapping a blood flow in a retrograde flow direction. By pre-shaping the leaflets 22A, 22B, 22C to cause the leaflets 22A, 22B, 22C to assume, e.g., a curved shape, a retrograde flow may cause the leaflets 22A, 22B, 22C to be pressed against each other such that a tightness in between the leaflets 22A, 22B, 22C may be improved in a closed position of the leaflets 22A, 22B, 22C. At the same time, a loading on the valvular support structure 20 may be reduced in that pressure forces acting on the leaflets 22A, 22B, 22C caused by a retrograde blood flow are substantially not or at least less redirected into radial forces acting on the valvular support structure 20, which otherwise may potentially cause pulling forces on the valvular support structure 20 causing the valvular support structure 20 to be pulled radially inwards.

A pre-shaping of the leaflets 22A, 22B, 22C may further cause a soft abutment of abutment edges of the leaflets 22A, 22B, 22C. In particular when the leaflets 22A, 22B, 22C move inwards to block a retrograde flow when the heart valve prosthesis is transferred from the open state to the closed state, the leaflets 22A, 22B, 22C may be caused to softly come into abutment with each other, hence avoiding a hard flapping of the leaflets 22A, 22B, 22C.

The flexible leaflet arrangement 21 forms, in between adjacent leaflets 22A, 22B, 22C, fastening sections 23 which serve to fixedly connect the flexible leaflet arrangement 21 to the valvular support structure 20. The fastening sections 23 may for example, as illustrated in FIG. 10, form flap sections 230, which may be folded to form for example a bag-like structure which may engage with end sections at the upper vertices 200 of the valvular support structure 20, or may be sewed to beam sections extending from the upper vertices 200 of the valvular support structure 20.

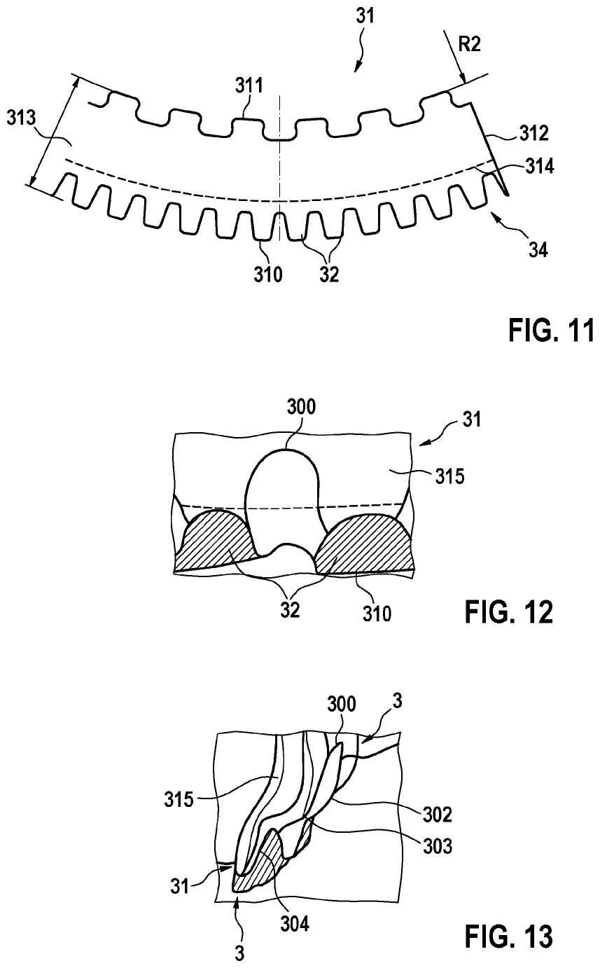

Referring now to FIG. 11, the skirt arrangement 31 of the anchoring member 3 may be formed from a single piece of flexible, pliable material 34, the piece of material 34 being sewed together at lateral edges 312, 313 to form a circumferentially closed structure.

In an alternative embodiment, the skirt arrangement 31 may be formed from multiple pieces of material 34, the pieces of material 34 being sewed together to again form a circumferentially closed structure.

Referring now to FIGS. 12 to 14, in one embodiment the anchoring support structure 30 may have a meandering shape being formed by radially outer portions 302 and radially inner portions 304 which are adjoined by linking portions 303. The radially outer portions 302 include the upper vertices 300 and are displaced radially with respect to the radially inner portions 304 towards the outside, the radially inner portions 304 including the lower vertices 301.

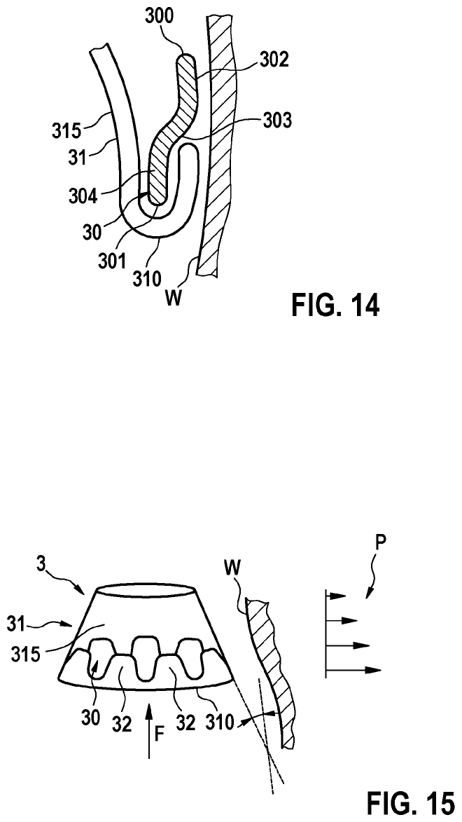

In this embodiment, the radially outer portions 302, which circumferentially alternate with the radially inner portions 304, may serve to provide for a seating of the anchoring member 3 on native structure at an implantation site, for example on a wall section W in the region of an annulus of an aortic valve of a patient, as illustrated in FIG. 14.

The radially inner portions 304, in turn, serve for supporting the flexible skirt arrangement 31, the flexible skirt arrangement 31 being fixed to the radially inner portions 304 by the folding sections 32, which are folded around the radially inner portions 304, as visible from FIG. 12.

As visible from FIG. 1 in combination with FIG. 15, the anchoring member 3 may have a generally conical shape, the anchoring member 3 tapering in the antegrade flow direction F and hence towards the valve member 2. As illustrated in FIG. 15, due to the conical shape a pressure P acting in between the anchoring member 3 and native structure, in particular a to wall section W at the implantation site, may vary along the antegrade flow direction X, the pressure P in between the anchoring member 3 and the wall section W being largest in the region of the inflow edge 310, wherein the distribution of the pressure P depends on the conical angle of the anchoring member 3.

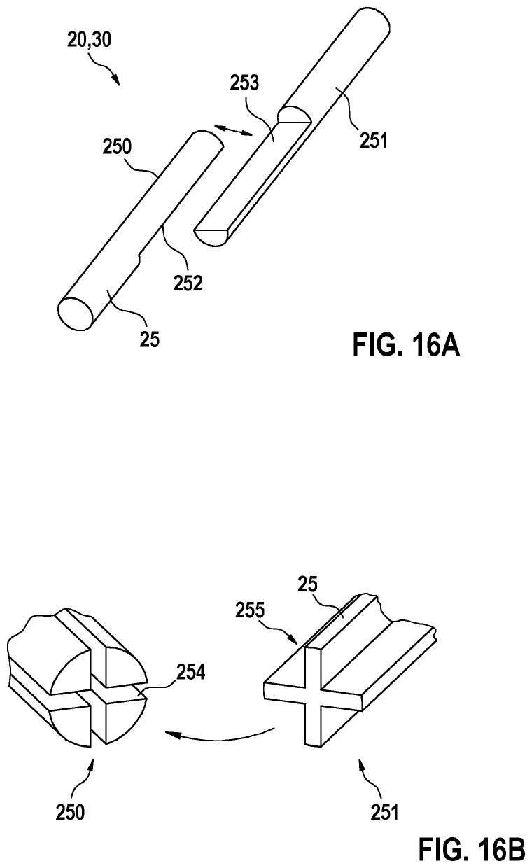

Referring now to FIG. 16A, the valvular support structure 20 as well as the anchoring support structure 30 may be formed by a wire 25 which may be bent to assume a meandering or crown-like shape, as visible from FIG. 4, respectively, FIG. 7. The valvular support structure 20 as well as the anchoring support structure 30 herein are formed by in each case a circumferentially closed ring, which for example may be formed by a wire 25 which continuously extends circumferentially to form a closed loop structure.

Herein, the wire 25 forming the valvular support structure 20 or the anchoring support structure 30 may be integrally closed in the circumferential direction. Alternatively, the wire 25 may have ends 250, 251 which, for forming the valvular support structure 20, respectively, the anchoring support structure 30, may be connected to each other for example by an adhesive bond, for example by gluing, soldering or welding, or by a positive-fit or press-fit connection, for example by an additional fitting sleeve or the like fitted on the wire 25. At the ends 250, 251 of the wire 25, flattened sections 252, 253 may be formed, such that the ends 250, 251 may be placed on one another and may be joined to each other without increasing the cross-sectional diameter of the wire 25.

An embodiment of a positive-fit connection of wire ends 250, 251 of a wire 25 is illustrated in FIG. 16B. For example, one wire end 250 of the wire 25 may include a slot arrangement 254, included for example of two perpendicular slots, into which the other wire end 251 may be inserted by a positive locking arrangement 255 having a cross shape. By engaging the wire ends 250, 251 with each other a positive locking connection in between the wire ends 250, 251 may be obtained, wherein the engagement in between the wire ends 250, 251 may in addition form a press-fit connection such that the wire ends 250, 251 are securely held on each other.

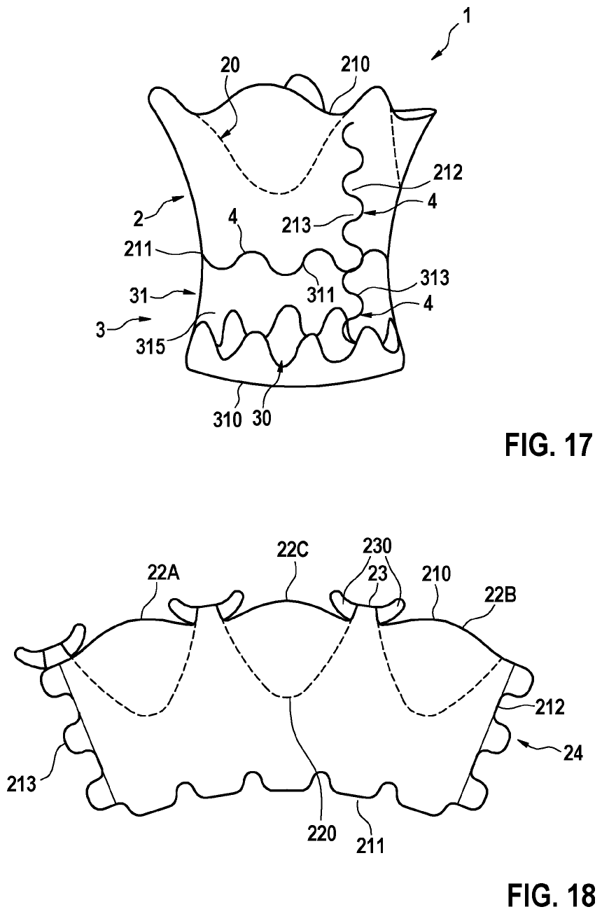

to Referring now to FIG. 17, the valve member 2 and the anchoring member 3 are interconnected by a seam 4 for fixing the flexible leaflet arrangement 21 of the valve member 2 to the flexible skirt arrangement 31 of the anchoring member 3. In addition, one or multiple seams 4 are formed on the flexible leaflet arrangement 21, respectively, the flexible skirt arrangement 31 in order to form in each case a circumferentially closed structure by one or multiple pieces of material for forming the valve member 2, respectively, the anchoring member 3.

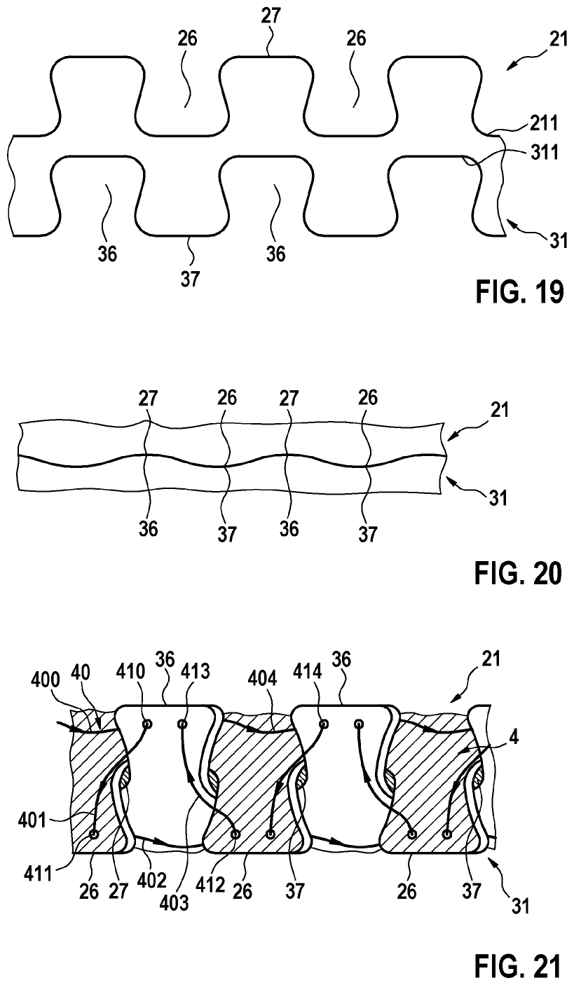

Referring now to FIG. 18, a piece of material 24 forming the flexible leaflet arrangement 21 may, at a joining edge 211 as well as at lateral edges 212, 213, have a meandering shape formed by, as visible in FIG. 19, joining flaps 26 alternating with recesses 27.

Just as well, a piece of material 34 forming the flexible skirt arrangement 31, as illustrated in FIG. 11, at a joining edge 311 as well as at lateral edges 212, 213 may have a meandering shape formed by joining flaps 36 alternating with recesses 37, as also visible in FIG. 19.

Referring now to FIGS. 19 and 20, in one embodiment, the flexible leaflet arrangement 21 of the valve member 2 and the flexible skirt arrangement 31 of the anchoring member 3—after forming the circumferentially closed structure of the flexible leaflet arrangement 21 and the flexible skirt arrangement 31 and after fixing the flexible leaflet arrangement 21 and the flexible skirt arrangement 31 to the valvular support structure 20, respectively, the anchoring support structure 30—are connected to each other by arranging the valve member 2 and the anchoring member 3 on each other such that the joining flaps 26, 36 of the flexible leaflet arrangement 21 and the flexible skirt arrangement 31 interleave with each other. The joining flaps 26 of the flexible leaflet arrangement 21 hence engage with recesses 37 of the flexible skirt arrangement 31 and the joining flaps 36 of the flexible skirt arrangement 31 engage with the recesses 27 of the flexible leaflet arrangement 21, as shown in FIG. 20.

Herein, in one embodiment, the joining flaps 26, 36 are arranged on one another to interleave with one another such that the joining flaps 26, 36 are arranged at an outer side of the heart valve prosthesis 1, the joining flaps 26, 36 coming to rest at an outside of a body 214 of the flexible leaflet arrangement 21 and a body 315 of the flexible skirt arrangement 31, as this is visible from FIG. 20 in view of FIGS. 21 and 23. In this way, the interleaving joining flaps 26, 36 may provide for a fixed, tight connection in between the valve member 2 and the anchoring member 3, without, however, interfering with a flow path inside of the heart valve prosthesis 1 and hence without altering a flow through the heart valve prosthesis 1.

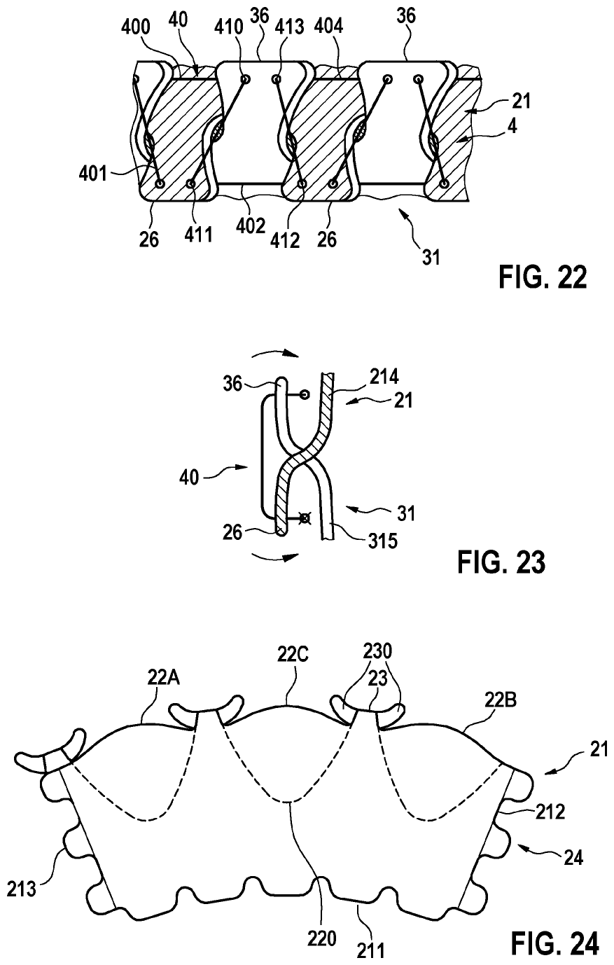

Referring now to FIGS. 21 to 23, a seam 4 for interconnecting the valve member 2 and the anchoring member 3 is formed by a thread 40 forming thread sections 400-404, the seam 4 having an opened trapezoidal shape. Herein, in contrast to FIG. 20 which shows the flexible leaflet arrangement 21 and the flexible skirt arrangement 31 from the inside of the heart valve prosthesis 1, FIGS. 21 and 22 show the flexible leaflet arrangement 21 and the flexible skirt arrangement 31 at the location of the seam 4 from the outside of the heart valve prosthesis 1. The seam 4 is solely formed on the outside and, as visible from FIG. 20, does not extend to the inside and hence does not interfere with a blood flow on the inside of the heart valve prosthesis 1.

Whereas FIG. 21 shows the seam 4 with a thread 40 forming the seam 4 in a loose state, FIG. 22 shows the seam 4 with the thread 40 forming the seam 4 being tightened, as it is the case in an assembled state of the heart valve prosthesis 1.

In one embodiment, transverse thread portions 400, 402, 404 extend in between neighbouring joining flaps 26, 36, each thread portion 400, 402, 404 being placed at an outside of the heart valve prosthesis 1, the thread portions 400, 402, 404 extending in parallel to one another as visible from FIGS. 21 and 22.

Each transverse thread portion 400, 402, 404 connects alike joining flaps 26, 36. In particular, thread portion 402 extends in between two adjacent joining flaps 26 of the flexible leaflet arrangement 21. Thread portion 404 extends in between two adjacent joining flaps 36 of the flexible skirt arrangement 31.

Each transverse thread portion 400, 402, 404 is adjoined by oblique thread portions 401, 403, the oblique thread portions 401, 403 extending likewise at the outside of the flexible leaflet arrangement 21 and the flexible skirt arrangement 31, as visible from FIGS. 21 and 22, such that the entire thread 40 forming the seam 4 comes to rest outside of the inner lumen of the heart valve prosthesis 1. Herein, thread portion 400 extends towards a thread opening 410 formed in a flap section 36, extends through the thread opening 410 and is adjoined by thread portion 401, which extends across the outside of the associated flap sections 36, 26 towards a thread opening 411 formed in the flap section 26 and through the thread opening 411 and is adjoined by thread portion 402, which extends across neighbouring joining flaps 26 at the outside of the flexible leaflet arrangement 21 and the flexible skirt arrangement 31 towards a thread opening 412 formed in the neighbouring flap section 26. At the thread opening 412, the thread 40 passes towards the outside of the flap section 26, as visible in FIG. 21, thread portion 403 extending at the outside of the heart valve prosthesis 1 towards thread opening 413 formed in the associated flap section 36. The thread 40 extends through the thread opening 413, such that thread portion 404 extends at the outside between neighbouring flaps 36 towards thread opening 414, and so on.

Due to the formation of the seam 4 by the thread 40, oblique portions 401, 403 as well as transverse portions 400, 402, 404 are placed at the outside of the flexible leaflet arrangement 21 and the flexible skirt arrangement 31, hence allowing for an unaltered flow through the heart valve prosthesis 1, because the seam 4 does not interfere with a flow at the inside of the heart valve prosthesis 1.

In addition, due to the formation of the seam 4 and because the flap sections 26, 36 are placed at the outside and generally may abut with tissue surrounding the heart valve prosthesis 1, the joining flaps 26, 36 may tightly abut with the body 315 of the flexible skirt arrangement 31, respectively, the body 214 of the flexible leaflet arrangement 21, such that the joining flaps 26, 36 provide for a fixed interconnection while preventing an irregular bulging of material at the location of the seam 4.

Referring now to FIGS. 24 to 41, for manufacturing the heart valve prosthesis 1 the valve member 2 and the anchoring member 3 may be formed first at separate units, and may then be mounted on each other to complete the heart valve prosthesis 1.

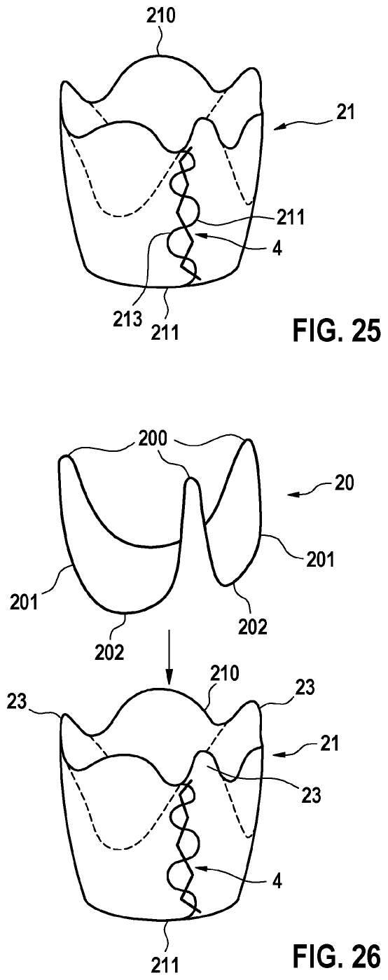

Referring first to FIGS. 24 to 32, for manufacturing the valve member 2 the circumferentially closed structure of the flexible leaflet arrangement 21 is formed for example from a single piece of material 24 forming the leaflets 22A, 22B, 22C (FIG. 24) by forming a seam 4 at edges 212, 213 (FIG. 25).

Once the closed structure of the flexible leaflet arrangement 21 is formed, the valvular support structure 20 is placed inside the flexible leaflet arrangement 21 (FIG. 26), the valvular support structure 20 being received within the flexible leaflet arrangement 21 such that the valvular support structure 20 comes to rest with upper vertices 200 in the region of the fastening sections 23 of the flexible leaflet arrangement 21.

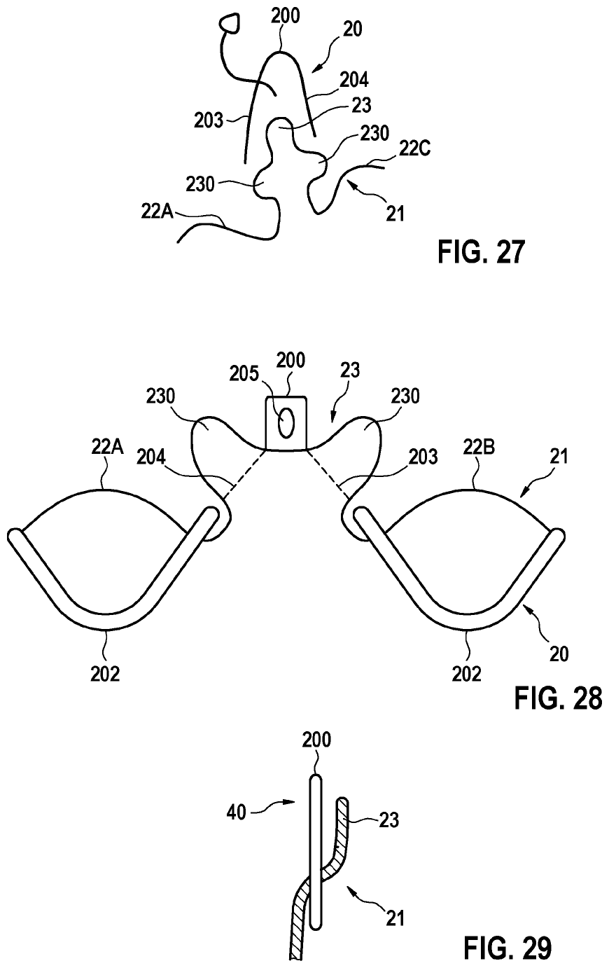

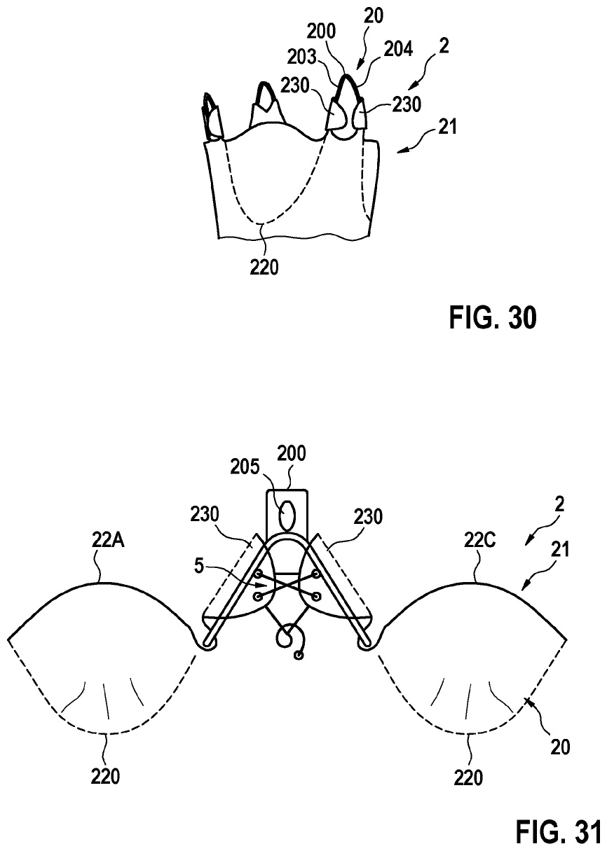

Subsequently, as illustrated in FIGS. 27 to 32, the fastening sections 23 are fixed to the associated tip sections formed at the upper vertices 200 in that each fastening section 23, from the outside, is inserted through beam sections 203, 204 adjoining the associated upper vertex 200 (FIG. 27) in order to then fold the flap sections 230 of the fastening section 23 back towards the inside, as illustrated in FIGS. 28 to 30. The flap sections 230 hence, as visible from FIG. 30, reach to the outside of the tip sections formed by the upper vertices 200 and the adjoining beam sections 203, 204 of the valvular support structure 20, allowing hence to sew together the flap sections 230 by means of, for example, a cross-shaped seam connection 5, as illustrated in FIG. 31.

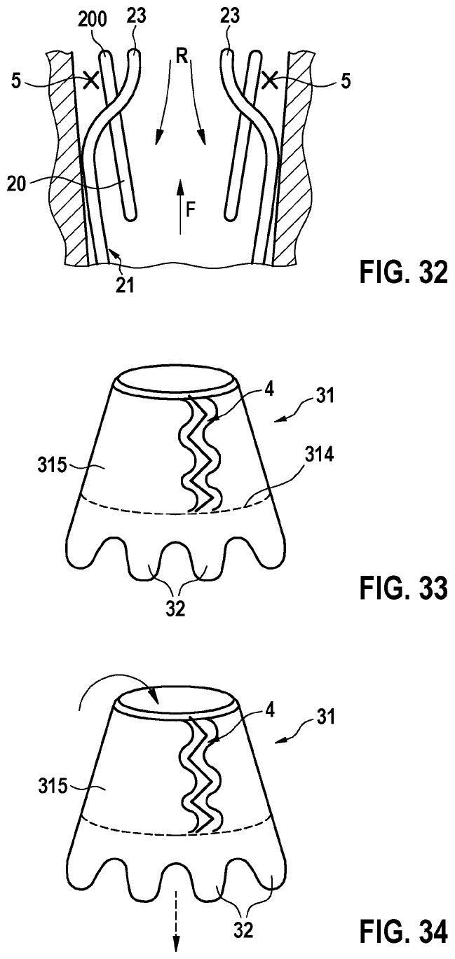

The seam connection 5 hence, as illustrated in FIG. 32, comes to rest outside of the valvular support structure 20, and hence is arranged outside of a flow path formed within the valve member 2, such that the seam connection 5 does not interfere with a flow through the valve member 2.

Referring now to FIGS. 33 to 40, for manufacturing the anchoring member 3 the circumferentially closed structure of the skirt arrangement 31 is formed, for example, from a single piece of material by sewing lateral edges of the skirt arrangement 31 together to form a seam 4 (FIG. 33).

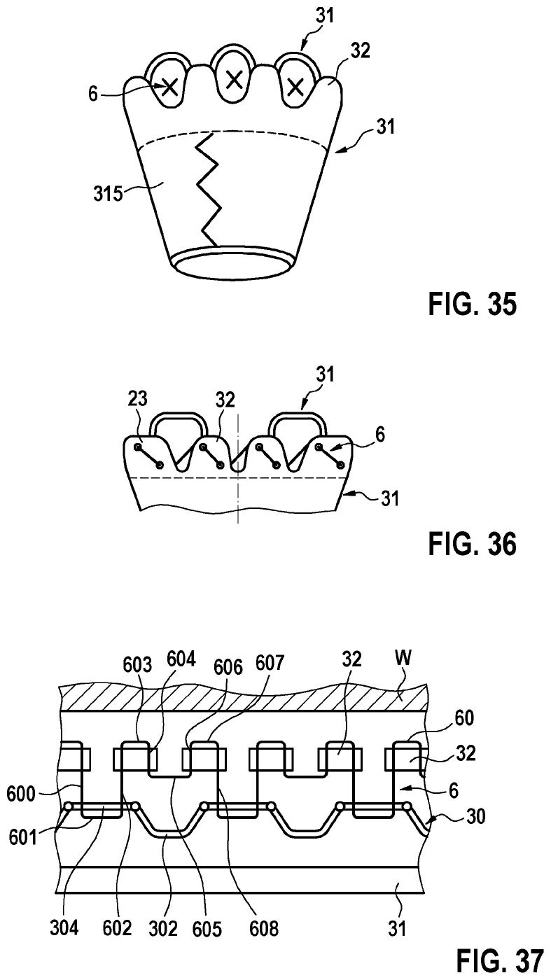

The flexible skirt arrangement 31 now is turned inside-out (FIG. 34) in order to fix the anchoring support structure 30 to the folding sections 32 of the flexible skirt arrangement 31 (FIG. 35).

The fixation of the anchoring support structure 30 to the folding sections 32 of the flexible skirt arrangement 31 takes place by a seam 6 which, as schematically shown in FIG. 37, interconnects the anchoring support structure 30 to the folding sections 32.

As visible from FIG. 37, the seam 6 is formed by a thread 60 forming thread sections 600-608.

In particular, a thread section 600 extends from a folding section 32 towards the anchoring support structure 30. An adjoining thread section 601 extends around the anchoring support structure 30, and a thread section 602 extends back towards a neighbouring folding section 32. A thread section 603 extends across the folding section 32, a thread section 604 extends through the folding section 32, a thread section 605 extends across towards another, neighbouring folding section 32, a thread section 606 extends through the folding section 32 towards the outside, a thread section 607 extends across the respective folding section 32, and a thread section 608 extends through the folding section 32 and towards another section of the anchoring support structure 30. In this way, the anchoring support structure 30 is fixedly connected to the folding sections 32 and in this way to the flexible skirt arrangement 31.

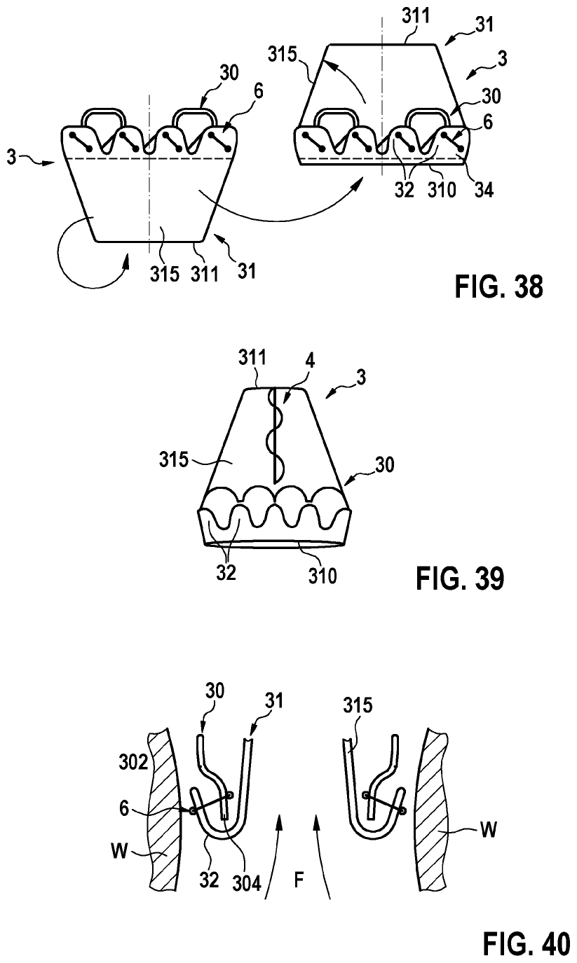

As visible from FIG. 38, now the flexible skirt arrangement 31 with the anchoring support structure 30 fixed thereto is folded back in that the body 315 is turned outside-in through the anchoring support structure 30 such that the anchoring member 3 is formed, as shown in FIG. 39.

In this way, the anchoring support structure 30, at the radially inner portions 304, by a seam connection 6 is connected to the flexible skirt arrangement 31. The anchoring support structure 30 herein is sewed at the radially inner portions 304 to the folding sections 32 folded with respect to the body 315 of the flexible skirt arrangement 31 to reach around the radially inner portions 304 of the anchoring support structure 30, as visible from FIG. 40.

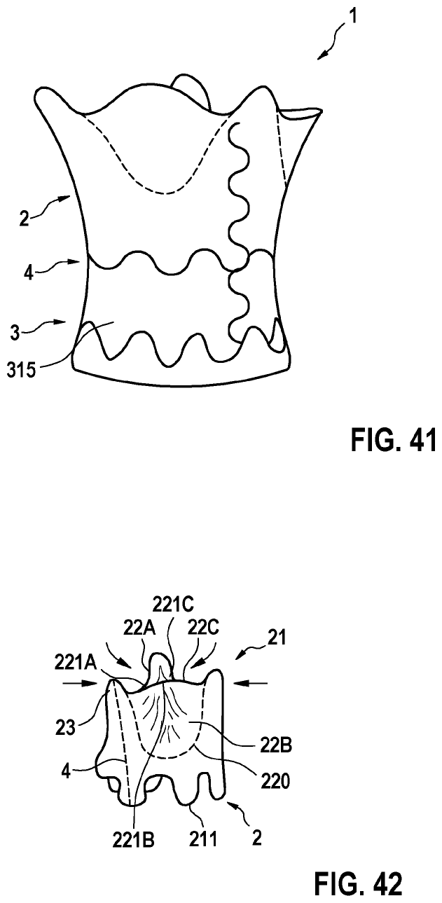

By now connecting the valve member 2 and the anchoring member 3 to one another by the seam 4, the heart valve prosthesis 1 is formed, as shown in FIG. 41, the heart valve prosthesis 1 being deployable by a delivery catheter in that the heart valve prosthesis 1 may be received within the delivery catheter in a radially compressed state and may be deployed by self-expansion at an implantation site to engage with native structure at the implantation site.

Referring now to FIGS. 42 to 47, there is a general desire to adapt a valve member 2 of a heart valve prosthesis 1 such that it may be beneficially received at an implantation site, for example at the location of a natural aortic valve in order to replace the natural aortic valve. As the aorta at the location of the natural aortic valve generally does not have a circular cross-sectional shape, but substantially differs from such circular cross-sectional shape, it may generally be beneficial to provide a heart valve prosthesis 1 which likewise is not circular in cross section, but is adapted to more closely match the actual shape of a native aorta.

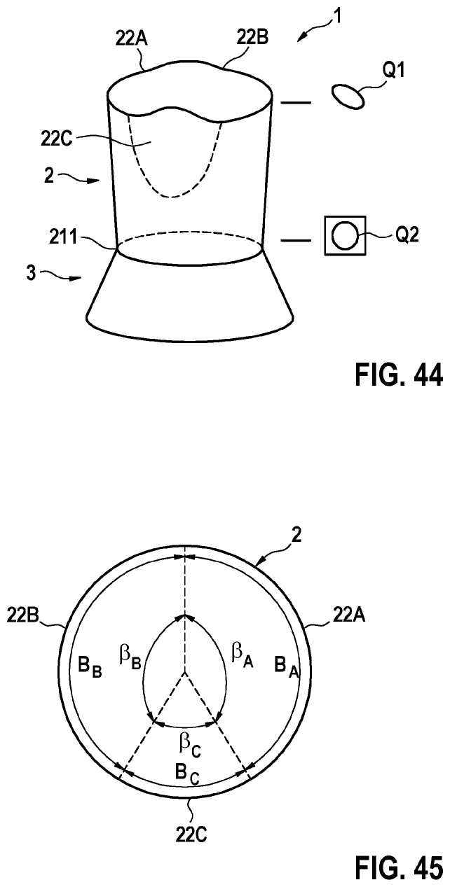

For this, as visible from FIG. 44, the valve member 2 of the heart valve prosthesis 1, in particular in the regions of the leaflets 22A-22C, may have a cross-sectional shape Q1 which for example may be elliptical, whereas in another region, for example at a waist in between the valve member 2 and the anchoring member 3 the cross-sectional shape Q2 may be circular. Hence, in particular in the region of the leaflets 22A-22C, the shape of the heart valve prosthesis 1 is formed to differ from a circular shape, such that in the region of the leaflets 22A-22C the heart valve prosthesis 1 may be adapted to a non-circular shape of the aorta.

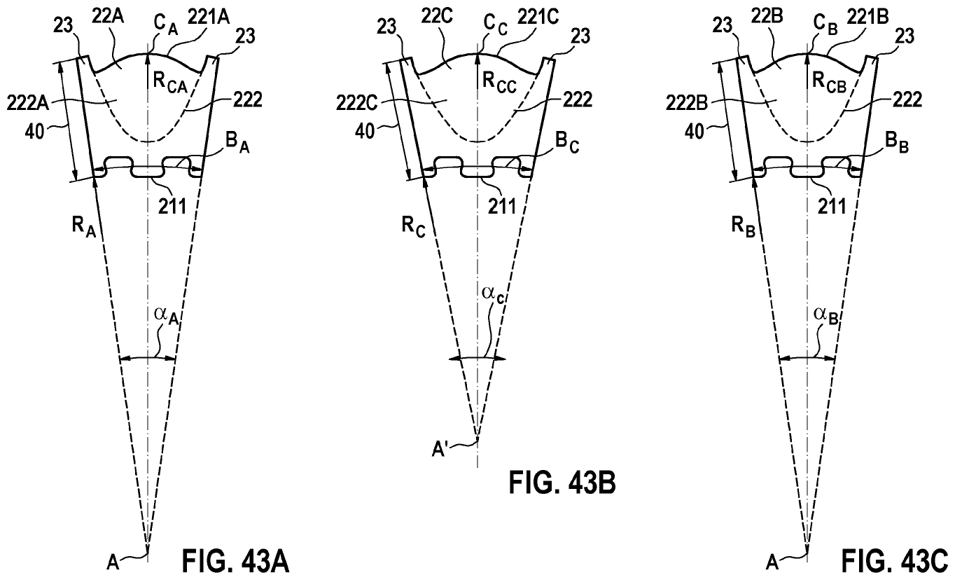

To form the heart valve prosthesis 1 in an asymmetric, non-circular way, the different leaflets 22A-22C may be formed differently, as visible from FIG. 42 in view of FIGS. 43A-43C. Herein, the leaflets 22A-22C may be formed by different pieces of material, as visible from FIGS. 43A-43C (i.e. FIGS. 43A-C), where it also is conceivable, that the different leaflets 22A-22C are formed by a single piece of material, similar to the embodiment of FIG. 8 as described above.

Generally, the different leaflets 22A-22C are formed differently. In particular, as visible from FIG. 45, the leaflets 22A-22C are formed such that they extend across different opening angles β_{A}, β_{B}, β_{C}. In particular, first leaflets 22A, 22B may span opening angles β_{A}, β_{B} which are equal to one another, whereas a second leaflet 22C may span an opening angle β_{C} which is differs from the opening angles β_{A}, β_{B} of the leaflets 22A, 22B. In particular, the opening angle β_{C}, as visible in FIG. 45, may be smaller than the opening angles β_{A}, β_{B} of the leaflets 22A, 22B.

The asymmetric shaping of the different leaflets 22A-22C may in particular be achieved by forming the pieces making up the leaflets 22A-22C in a different manner. In particular, a radius R_{A}, R_{B}, R_{C} of an inner arc B_{A}, B_{B}, B_{C} of the single pieces may differ as visible from FIGS. 43A, 43B, 43C. In addition, an angle α_{A}, α_{B}, α_{C} across which the single pieces extend to make up the conical shape of the flexible leaflet arrangement 21 may differ.

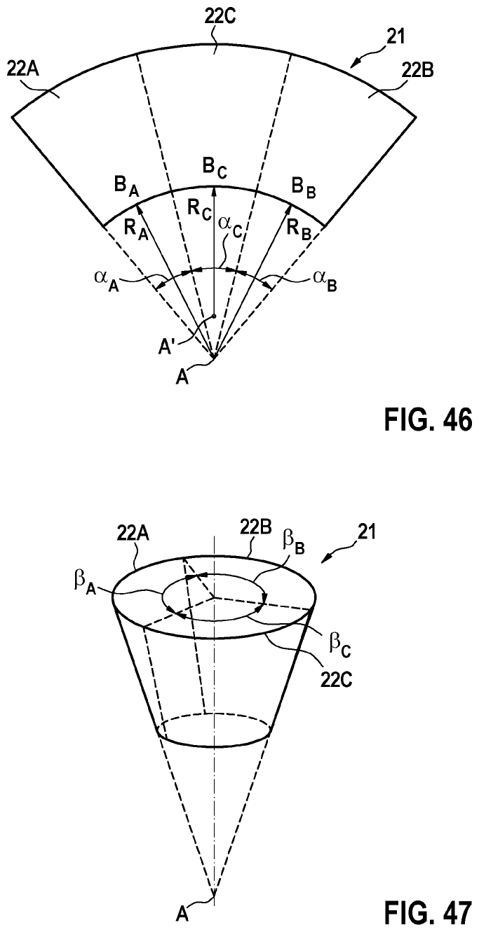

Generally, as schematically shown in FIGS. 46 and 47, a conical leaflet arrangement 21 is made up by a structure which, when arranged flatly in a two-dimensional plane as visible in FIG. 46, has the shape as shown in FIG. 46 and in particular corresponds to a section of a ring. Each piece associated with a leaflet 22A-22C extends across an angle of the α_{A}, α_{B}, α_{C}, wherein, as shown in FIGS. 43A-43C, the angles of α_{A}, α_{B}, α_{C} may differ. Also, to obtain the asymmetric shape, the radius R_{A}, R_{B}, R_{C} of an inner arc B_{A}, B_{B}, B_{C} of the pieces associated with the leaflets 22A-22C may differ, as visible from FIG. 46 in view of FIGS. 43A-43C. In particular the radius R_{A}, R_{B} of the pieces associated with leaflets 22A, 22B may correspond to the distance between the inner arc B_{A}, B_{B} and the (virtual) apex A of the conus of the leaflet arrangement as shown in FIG. 47, whereas the radius R_{C} of the piece forming the leaflet 22C is associated with a different reference point A′ and hence differs.

In addition to the asymmetric shaping to cause the leaflets 22A, 22B, 22C to span different opening angles β_{A}, β_{B}, β_{C}, the leaflets 22A, 22B, 22C at free edges 221A, 221B, 221C include different curvatures. Namely, at a centre point C_{A}, C_{B}, C_{C} in between fastening sections 23 bounding the free edges 221A, 221B, 221C the free edges 221A, 221B, 221C include different curvatures, defined by different radii of curvature R_{CA}, R_{CB}, R_{CC} as visible from FIGS. 43A-43C. In particular, the free edge 221C of the leaflet 22C may include a larger radius of curvature R_{CC} and hence a smaller curvature in comparison to the leaflets 22A, 22B.

In this way, an asymmetrical shape in particular at the leaflet 22C may be caused, as visible is from FIG. 44.

The invention is not limited to the embodiments described above but may be implemented in an entirely different fashion.

A heart valve prosthesis of the kind described above may be used as a prosthetic valve replacement at an aortic valve of a patient, but in principle may also be used as a valve at a different location. For example, the heart valve prosthesis may be used as a mitral valve prosthesis or a tricuspid valve prosthesis.

Structure, design and shape of a valve member and an anchoring member may differ from the embodiments described above. In particular, different numbers of leaflets may be used on a valve member, and accordingly a shape of a valvular support structure may differ from embodiments described above. Likewise, a design of an anchoring support structure may differ from embodiments described above.

A heart valve prosthesis having leaflets of different geometric shape and hence causing an asymmetric shape of a valve member of the heart valve prosthesis may be used in connection with an anchoring member as described in different embodiments above, but may also be used without an anchoring member of the kind as described above. In particular, an asymmetrically shaped valve member may also be used in a heart valve prosthesis having no separate support structures, but having a single support structure, for example in the shape of a stent included of structurally connected portions.

### LIST OF REFERENCE NUMERALS

1 Heart valve prosthesis

2 Valve member

20 Valvular support structure (support ring)

200 Tip section (Upper vertex)

201 Curved section

202 Lower vertex

203, 204 Beam section

205 Eyelet

21 Leaflet arrangement

210 Outflow edge

211 Joining edge

212, 213 Edge

214 Body

22A, 22B, 22C Leaflet

220 Bending line

221A, 221B, 221C Free edge

222A, 222B, 222C Leaflet area

23 Fastening section

230 Flap section

24 Material

25 Wire

250, 251 End

252, 253 Flattened section

254 Slot arrangement

255 Positive-locking arrangement

26 Joining flap

27 Recess

3 Anchoring member

30 Anchoring support structure

300 Upper vertex

301 Lower vertex

302 Outer portion

303 Linking portion

304 Inner portion

31 Skirt arrangement

310 Inflow edge

311 Joining edge

312, 313 Edge

314 Fold line

315 Body

32 Folding section

34 Material

36 Flap section

37 Recess

4 Seam

40 Thread

400-404 Thread portion

410-414 Thread opening

5 Seam

6 Seam

60 Thread

600-604 Thread portion

A Apex of cone

A′ Reference point

α_{A}, α_{B}, α_{C} Angle

β_{A}, β_{B}, β_{C} Opening angle

B_{A}, B_{B}, B_{C} Arc length

F Antegrade flow direction

P Pressure distribution

Q1, Q2 Cross-sectional shape

R Retrograde flow direction

R_{A}, R_{B}, R_{C} Radius

R_{CA}, R_{CB}, R_{CC} Radius of curvature

W (Aortic) wall section

While preferred embodiments have been described, it should be understood that other modifications, substitutions and alternatives are apparent to one of ordinary skill in the art. Such modifications, substitutions and alternatives can be made without departing from the spirit and scope of the invention, which should be determined from the appended claims. Various features of the invention are set forth in the appended claims.

1. A heart valve prosthesis, comprising:
a valvular support structure, a plurality of flexible leaflets supported by the valvular support structure, the flexible leaflets being movable between an opened position to permit a blood flow in an antegrade flow direction (F) and a closed position to block a blood flow in a retrograde flow direction (R), wherein each flexible leaflet forms a free edge, the free edges being configured to abut one another in the closed position;
and wherein,
when viewed in a cross-sectional plane transverse to the antegrade flow direction (F), a first of the flexible leaflets extends across a first opening angle (β_{A}, β_{B}) and a second of the flexible leaflets extends across a second opening angle (β_{C}) different from the first opening angle (β_{A}, β_{B}), wherein the free edge of the first of the flexible leaflets comprises a first convex curvature and the free edge of the second of the flexible leaflets comprises a second convex curvature different than the first convex curvature.

2. The heart valve prosthesis of claim 1, wherein the second opening angle (β_{C}) is smaller than the first opening angle (β_{A}, β_{B}).

3. The heart valve prosthesis of claim 1, comprising multiple first flexible leaflets each extending across said first opening angle (β_{A}, β_{B}).

4. The heart valve prosthesis of claim 1, wherein the second convex curvature is smaller than the first convex curvature (R_{CA}, R_{CB})

5. The heart valve prosthesis of claim 1, wherein the flexible leaflets are formed from separate pieces of material, wherein a geometric shape of a piece of material forming the first of the flexible leaflets differs from a geometric shape of a piece of material forming the second of the flexible leaflets.

6. The heart valve prosthesis of claim 1, wherein the flexible leaflets form an arrangement that is conically tapered in the retrograde flow direction (R).

7. The heart valve prosthesis of claim 1, wherein the valvular support structure comprises a ring.

8. The heart valve prosthesis of claim 7, wherein the ring comprises a plurality of curved sections, each curved section being associated with one of the plurality of flexible leaflets to define a bending line about which the associated flexible leaflet is flexibly bendable to permit movement between the opened position and the closed position.

9. The heart valve prosthesis of claim 1, wherein the valvular support structure forms a plurality of tip sections that fix the flexible leaflets.

10. The heart valve prosthesis of claim 1, comprising an anchoring member, the anchoring member comprising a flexible skirt and an anchoring support structure that supports the flexible skirt, wherein the valvular support structure and the anchoring support structure are structurally separate from one another, but are flexibly connected to each other via the flexible leaflets and the flexible skirt.

11. The heart valve prosthesis of claim 10, wherein the flexible skirt forms an inflow edge, wherein at least a portion of the flexible skirt is conically tapered in the antegrade flow direction (F) beyond the inflow edge.

12. The heart valve prosthesis of claim 10, wherein the anchoring support structure comprises a ring.

13. The heart valve prosthesis of claim 12, wherein the ring has a meandering shape.

14. The heart valve prosthesis of claim 12, wherein the flexible leaflets form an arrangement comprising a first joining edge, and the flexible skirt comprises a second joining edge, wherein the first joining edge and the second joining edge are connected to each other by a seam.

15. The heart valve prosthesis of claim 14, wherein the arrangement, at the first joining edge, forms at least one first joining flap and/or the flexible skirt, at the second joining edge, forms at least one second joining flap.
